(19) United States
(12) Patent Application Publication
(10) Pub. No.: US 2024/0404270 A1
(43) Pub. Date: Dec. 5, 2024

(54) AI-POWERED DEVICES AND METHODS TO PROVIDE IMAGE AND SENSOR INFORMED EARLY WARNING OF CHANGES IN HEALTH

(71) Applicant: NEW YORK UNIVERSITY, New York, NY (US)

(72) Inventors: Daniel K. Sodickson, Larchmont, NY (US); Hersh Chandarana, Scarsdale, NY (US); Sumit Chopra, New York, NY (US)

(73) Assignee: NEW YORK UNIVERSITY, New York, NY (US)

(21) Appl. No.: 18/806,626

(22) Filed: Aug. 15, 2024

Related U.S. Application Data
(63) Continuation-in-part of application No. PCT/US2023/013257, filed on Feb. 16, 2023.
(60) Provisional application No. 63/310,975, filed on Feb. 16, 2022.

(51) Int. Cl. G06V 10/82; G06V 10/774; G16H 30/40

(52) U.S. Cl. CPC G06V 10/82; G06V 10/774; G16H 30/40; G06V 2201/03

(57) ABSTRACT

One embodiment relates to a method that includes receiving input data. The input data includes data from a plurality of modalities including imaging or sensing modalities. The method includes processing the input data with a neural network trained to identify shared characteristics among the plurality of modalities. The neural network distills the input data to generate a representation. The method includes processing the representation with the neural network to generate an output.

### CROSS-REFERENCE TO RELATED APPLICATIONS

The present application is a continuation-in-part (CIP) of PCT Application PCT/US2023/013257, filed Feb. 16, 2023, and claims the benefit of and priority to U.S. Provisional Patent Application No. 63/310,975, filed Feb. 16, 2022, the entire contents of which are incorporated herein by reference.

### BACKGROUND

The present application relates generally to an imaging or sensing system. More specifically, the present application relates to an imaging or sensing system using artificial intelligence to combine information from various imaging or sensing modalities and/or various imaging or sensing sessions to generate early warning of changes in health.

Imaging systems are used to acquire images of patients, organisms, or materials. Many different modes of imaging may be used to acquire images of patients. Medical imaging is used routinely to diagnose disease, to guide therapy, and/or to monitor the progress of disease in high-risk individuals. For example, imaging systems may include Magnetic Resonance Imaging (MRI), Computed Tomography (CT), Positron Emission Tomography (PET), Nuclear Imaging, ultrasound, and X-ray, among others, in addition to assorted optical camera systems. Imaging systems are highly valuable tools in research and clinical care.

Image quality may vary depending on the imaging system. Particularly for cross-sectional imaging devices like MRI, CT, and PET, there is generally a trade-off with cost increasing, mobility decreasing, and system size increasing as image quality increases. Further, image quality generally increases as imaging “power,” such as magnetic field strength in MRI, increases, and also as imaging time, such as duration of patient exposure to the imaging system for image capture, increases.

Because of a focus on the need to acquire high quality images directly from imaging a patient with a device, many imaging systems are expensive, slow, difficult to access, and/or require a bulky apparatus. For example, MRI imaging systems may cost millions of dollars, require minutes to hours to acquire images, occupy dozens of feet of floor space, and/or only be available at hospitals, outpatient centers, or research facilities.

As a result of this focus on high-quality images driven by large, stationary imaging systems, such imaging systems tend to be immobile and located at hospital or research facilities. As the infrastructure to support such large imaging systems is extensive, and the capital cost of such systems is high, imaging systems are spatially sparse, that is, there is a low density of imaging systems relative to either population or land mass. In addition, the focus on generation of high quality images results in the collection of imaging data from any given patient tending to occur infrequently, such as only upon visits to doctors. Thus, overall imaging data for a given patient tends to be spatially and temporally sparse.

A need exists for improved technology, namely, for affordable, faster, more frequent, higher-quality, and/or physically easily-accessible imaging systems that can be applied to a wide range of contexts for imaging patients. More accessible imaging would also be of value for preventative maintenance of devices and monitoring of the function of other complex systems over time.

Sensing systems are used to acquire data relating to a patient. Wearable and/or environmental sensors are being explored increasingly for frequent or even continuous monitoring of people and/or systems. Many different modes of sensing are currently available, and new sensors are being developed all the time. Sensing systems may include chemical sensors, sensors of physical properties such as pressure, temperature, impedance, or mechanical strain, and sensors of various other properties that may characterize bodies or systems. Some examples include, but are not limited to, bioimpedance sensors, skin conductance sensors, electrocardiograms (EKGs), electromyograms (EMGs), electroencephalograms (EEGs), radar sensors, near infrared (NIR) sensors, and accelerometers. As compared with imaging systems (which may be composed of arrays of carefully-coordinated sensors), however, individual sensor systems designed for continuous monitoring do not generally probe the spatial organization of bodies or systems with high resolution.

In addition to improving the accessibility of imaging systems, it is beneficial to combine information from sporadic imaging sessions using high resolution imaging systems with information from more continuous sensing systems. However, imaging and sensing data generally have different characteristics, and may be difficult to combine. In particular, it is difficult to assemble a multifaceted characterization of ongoing health (e.g., the health of humans, other organisms, or complex systems) over time from disparate sources, like imaging and sensing systems. Even for a single source, subtle and multifactorial changes over time can be difficult to characterize.

### SUMMARY

One embodiment of the present disclosure relates to a method that includes receiving input data. The input data includes data from a plurality of modalities including imaging or sensing modalities. The method includes processing the input data with a neural network trained to identify shared characteristics among the plurality of modalities. The neural network distills the input data to generate a representation. The method includes processing the representation with the neural network to generate an output.

Another embodiment of the present disclosure relates to a system. The system includes a memory including instructions and at least one processor. The at least one processor executes the instructions to receive input data. The input data includes at least one of data from multiple imaging modalities, data from multiple sensing modalities, and data from multiple different timepoints. The at least one processor further executes the instructions to process the input data using a neural network to generate a representation and process the representation with the neural network to generate an output.

Another embodiment of the present disclosure relates to a non-transitory processor-readable medium storing code representing instructions to be executed by one or more processors. The instructions include code causing the one or more processors to receive input data. The input data includes at least one of data from multiple imaging modalities, data from multiple sensing modalities, and data from multiple different timepoints. The instructions include code causing the one or more processors to process the input data using a neural network to generate a representation and process the representation with the neural network to generate an output.

This summary is illustrative only and is not intended to be in any way limiting.

### BRIEF DESCRIPTION OF THE DRAWINGS

A clear conception of the advantages and features constituting the present disclosure, and of the construction and operation of typical mechanisms provided with the present disclosure, will become more readily apparent by referring to the exemplary, and therefore non-limiting, embodiments illustrated in the drawings accompanying and forming a part of this specification, wherein like reference numerals designate the same elements in the several views, and in which:

FIG. 1 is a schematic illustration of one embodiment of a Type 1 imaging system.

FIG. 2 is a schematic illustration of one embodiment of a Type 2 imaging system.

FIG. 3 is a schematic illustration of one embodiment of a Type 3 sensing system.

FIG. 4 is a method for processing monitoring device data, according to an embodiment.

FIG. 5 is a schematic illustration of one embodiment of a health diagnostic system configured to communicate with a Type 1, Type 2 and/or Type 3 imaging/sensing system.

FIG. 6A is a schematic illustration of a process of generating a digital representation from imaging data in an encoder neural network to assess a health risk, according to an embodiment. FIG. 6B is a schematic illustration of an example of the process in FIG. 6B, according to another embodiment.

FIG. 7 is a schematic illustration of generating a representation, such as that shown in FIGS. 1-3, by generating a shared representation connecting low-quality and high-quality imaging data using an autoencoder neural network, according to an embodiment.

FIG. 8 is a schematic illustration of generating a representation, such as that shown in FIGS. 1-3, by generating a shared representation connecting imaging data from different imaging modalities using an autoencoder neural network, according to an embodiment.

FIG. 9 is a schematic illustration of generating a representation, such as that shown in FIGS. 1-3, by generating a shared representation connecting data from different imaging or sensing modalities using a contrastive or Siamese neural network, according to an embodiment.

FIG. 10 is a schematic illustration of generating a representation, such as that shown in FIGS. 1-3, by transforming data from one imaging or sensing modality using representations shared with another imaging or sensing modality, and combining the transformed data from one modality with data from the other modality to generate a common representation used to assess a health risk, according to an embodiment.

FIG. 11 is a schematic illustration of combining shared representations from more than one imaging or sensing modality to assess a health risk, according to an embodiment.

FIG. 12 is a schematic illustration of combining shared representations from more than one imaging or sensing modality to generate improved images, combined images, and/or change maps, according to an embodiment.

FIG. 13 is a schematic illustration of obtaining shared representations from various imaging and sensing modalities at various points in time and combining the shared representations to assess the evolution of a health risk over time, according to an embodiment.

FIG. 14 is a schematic illustration of utilizing a cumulative set of shared representations to refine an assessment of a health risk and modify the acquisition of new imaging or sensing data, according to an embodiment.

FIG. 15 is a schematic illustration of generating a cumulative set of shared representations and utilizing the cumulative set of shared representations to generate improved images, combined images, and/or change maps, according to an embodiment.

FIG. 16 is a schematic illustration of cumulative representations from different imaging or sensing sessions, using various imaging or sensing modalities with various degrees of accessibility deployed at time intervals of various frequency, and the use of the cumulative representations to establish a health trajectory in which changes of concern are flagged, according to an embodiment.

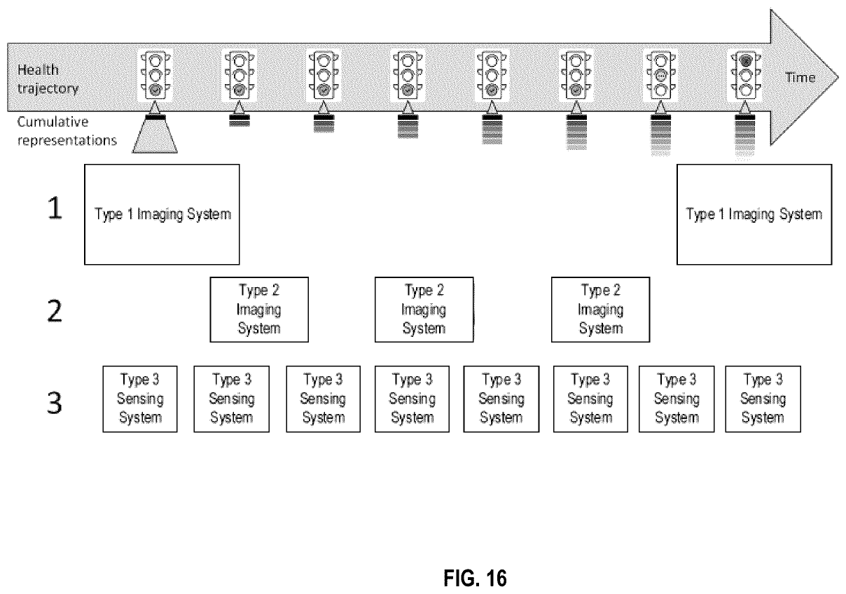

The foregoing and other features of the present disclosure will become apparent from the following description and appended claims, taken in conjunction with the accompanying drawings. Understanding that these drawings depict only several embodiments in accordance with the disclosure and are therefore, not to be considered limiting of its scope, the disclosure will be described with additional specificity and detail through use of the accompanying drawings.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

The present disclosure relates to the use of detailed imaging and more accessible sensing to provide robust tracking of health trajectories, and thus enable longitudinal monitoring and change detection. Longitudinal monitoring and change detection may be employed in medical applications, such personalized healthcare, as well as non-medical applications (for example, geology, climatology, astronomy, machine maintenance, ecosystem assessment). The present invention further provides robust updates to health trajectories using incomplete and/or imperfect data, such as that acquired using comparatively low-performance but highly accessible imaging technologies.

Incorporating information (e.g., inputs) from disparate imaging or sensing modalities and/or distinct imaging or sensing sessions is challenging. For example, diverse input information may include text records and sensor signals. These disparate inputs may have different size, dimension, and information content. Existing approaches have explored how to weigh, transform, combine, and process these disparate inputs. The present disclosure provides a framework of shared representations, and utilizes individual instances or cumulative sets of representations. As a result, the present disclosure provides a framework to assess risk, characterize change, improve image/signal quality, and/or establish trajectories of health, disease, or other function/dysfunction over time.

In particular, the present disclosure utilizes multiple tiers of imaging and sensing technology tied together by learned representations derived using artificial intelligence. The present disclosure provides a framework of shared representations among different imaging or sensing modalities and/or sessions. Shared representations generated from diverse imaging and sensing modalities, and/or cumulative representations generated over time, are used to establish individual baselines of health or normal functioning. Representations are evaluated over time to track ongoing states of health, to identify concerning departures from individual baselines, and to compare individual health trajectories to population norms. As a result, the invention described herein provides a robust and multifaceted early warning system for changes in health or normal functioning. This system may be used for managing human health, in addition to non-medical purposes, such as preventative maintenance of machines, and assessment of complex systems.

Referring to the figures generally, the present disclosure relates to an imaging and/or sensing system The system includes imaging and/or sensing devices, memory systems for storing or accessing sets of data acquired by those respective devices, artificial intelligences applied to the sets of data, and outputs resulting from applying the artificial intelligences to the sets of data. In some embodiments, the imaging and/or sensing system is configured to interact with a patient to generate a patient data set for the sensing system, where the generated patient data set may include a temporal component, for example, a MRI data set generated at a particular visit to a hospital having associated therewith a time stamp.

Referring to the figures generally, imaging and sensing technology, as discussed below, may be grouped into various “types”:
Type 1: State of the art cross-sectional imaging devices, such as MRI, CT, PET, or ultrasound. Generally, type 1 imaging systems provide the most robust imaging data. These medical imaging devices are expensive and resource intensive, and often require specialized expertise to interpret images. Generally, these devices are provided in hospitals or outpatient settings which are not widely accessible to the population. Regular monitoring with this technology is not currently practical.
Type 2: Lower-performance accessible imaging technologies with form factors that are not constrained to traditional imaging tubes. Such technologies can be deployed broadly in physicians' offices, in commercial settings like pharmacies or supermarkets, in places of work, and in personal homes. Such technologies could include low-field MRI machines, portable CT machines, and/or handheld ultrasound devices. Generally, type 2 imaging systems provide a less robust imaging data than type 1 imaging systems.
Type 3: Wearable and/or environmental sensors which can monitor health continuously or at regular intervals during daily life activities. Such technologies may include optical tracking devices in homes and/or physical/chemical/electrical sensors incorporated into clothing, or attached/implanted within individual bodies. Generally, type 3 imaging systems provide a less robust imaging data than type 2 imaging systems.

The distinction between each “type” can be thought of as a level of confidence that a clinician, such as a radiologist, may have in making assessment based on the resulting images. A clinician would feel comfortable in making an assessment from images from a Type 1 system. With Type 2 systems, a clinician would likely make an assessment, but would have a lower level of confidence given the reduced image quality as compared with Type 1. With Type 3 systems, a clinician would not be comfortable making an assessment given that Type 3 sensors do not deliver signals that clinicians are used to interpreting.

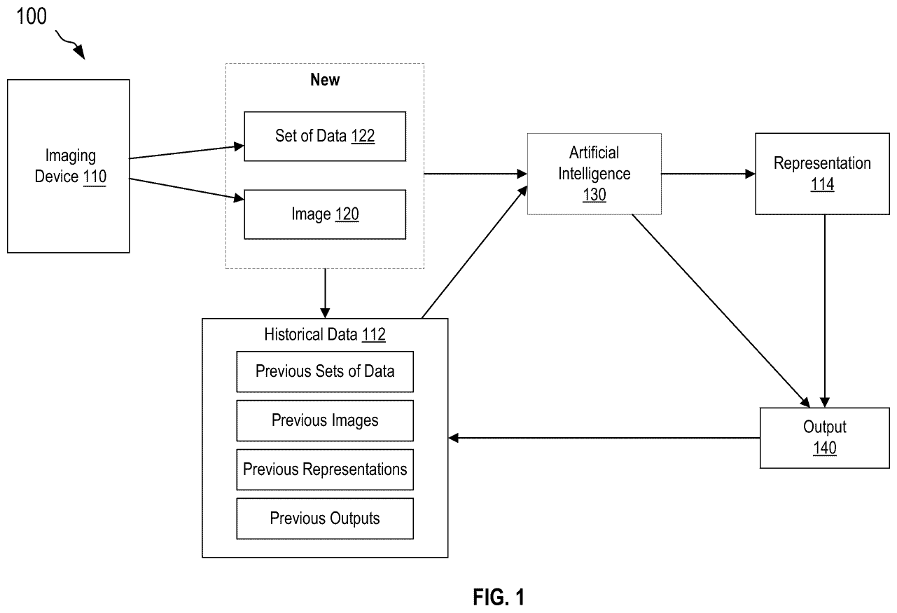

Referring to FIG. 1, an imaging system 100 is depicted according to an example embodiment. The imaging system 100 is a Type 1 imaging system that provides improved imaging in traditional radiology settings. Traditional radiology settings may include hospitals, outpatient imaging facilities, and image-aware physicians' offices. The imaging system 100 includes an imaging device 110, historical data 112, a representation 114, an image 120, a set of data 122, an artificial intelligence 130, and an output 140. The imaging system 100 is configured to use the artificial intelligence 130 to generate the representation 114 and the output 140, the output 140 providing an image-informed early warning of changes in the health of a patient. The imaging system 100 may be used repeatedly over time for an individual patient.

The imaging device 110 is a device that is configured to acquire images of a patient. In some examples, the imaging device 110 may be an MRI, CT, PET, Nuclear Imaging, ultrasound, X-ray machine, and/or a machine associated with another imaging modality. The image 120 is an image of a patient acquired via use of the imaging device 110. The set of data 122 may be information related to the image 120 or a separate image that shares properties with the image 120. The set of data 122 may be acquired by the imaging device 110 or a separate imaging device 110

The imaging system 100 further includes historical data 112. The historical data 112 includes previous information acquired by and/or created by the imaging system 100. For example, the historical data 112 may include previous images, sets of data, representations, and/or outputs. The artificial intelligence 130 is configured to use the historical data 112 to generate the representation 114, inform the acquisition of new images and/or set of data, and to allow for the detection of changes between images, sets of data, representations, and/or sub-combinations thereof over time.

The representation 114 is a model of an individual patient's baseline state of health. The representation 114 can take the form of a feature vector derived using the artificial intelligence 130, and generated from the image 120, and/or the set of data 122. The artificial intelligence 130 distills the image 120 and/or the set of data 122 into the representation 114. The representation 114 may be updated by the imaging system 100 when the patient undergoes a new imaging exam and a new image 120 and/or set of data 122 is acquired. An individual representation, or a cumulative set of representations generated over time may be used to assess risk, characterize change, improve image quality, and/or establish trajectories of health for a patient over time.

Multiple representations for an individual subject that vary over time are included in the historical data 112. As the historical data 112 allows for the usage of and comparisons to previous images, sets of data, and/or representations, updating a representation 114 with information from a new image 120 allows for the imaging system 100 to acquire a smaller quantity of time-consuming imaging data that makes up the image 120 than would otherwise be needed to generate a representation in the absence of previous images from the historical data 112. The representations of the patient in the imaging system 100 may also inform how the imaging system 100 is configured to acquire new images 120 and/or sets of data 122, and/or how the imaging system 100 is configured to generate new outputs 140. In some embodiments, foundation models trained on large volumes of imaging data are used to generate “starting” representations. In some embodiments, these “starting” representations may be used as inputs to classifiers, change detectors, and health warning systems.

The artificial intelligence 130 is configured to receive the image 120 and/or the set of data 122 to generate the representation 114 and output 140. The artificial intelligence 130 may be stored as part of the imaging device 110 or on a separate computing device. The artificial intelligence 130 may be trained on the historical data 112 including previous images, sets of data, representations, and/or outputs from the imaging system 100.

The artificial intelligence 130 may use various machine learning models to generate the representation 114 and output 140. In some embodiments, the artificial intelligence is trained using longitudinal imaging data. In some embodiments, the machine learning model includes representation learning to reduce high-dimensional data to low-dimensional data to better detect patterns and anomalies. In some embodiments, representation learning is supervised. In some embodiments, representation learning is self-supervised. In some embodiments, the machine learning model is a neural network. For example, the artificial intelligence 130 may use a convolutional neural network structure. In some embodiments, the artificial intelligence 130 may use an autoencoder network structure. In some embodiments, the artificial intelligence 130 may use a Siamese neural network structure.

The artificial intelligence 130 provides the output 140. The output may include automated image classification or interpretation, an imaged with increased speed, and/or improved imaging quality, among other outputs 140. The output 140 may then be provided back to the historical data 112 of the imaging system 100.

In some embodiments, the artificial intelligence 130 uses the representation 114 and previous representations in historical data 112 to generate a configuration for data acquisition for the imaging device 110 as an output 140. For example, a neural network can be used to learn which data points are most important, and these data points can be used to choose what types of data to acquire.

The output 140 may be an assessment of change, in which the artificial intelligence 130 assesses a new image 120, set of data 122, and/or representation 114 in comparison to previous images, sets of data, and representations from the historical data 112 to flag changes in individual subjects and/or to identify early trajectories toward known diseases or development of risk indicators. While existing technology allows for assessment of figure risk of disease, these solutions generally do not include longitudinal or historical data, or multimodal data. In some examples, known diseases may include cancer or a neurodegenerative disease. This allows for automatic assessment to detect of change over time, and detection of subtle shifts that may not be discernible by a radiologist.

In another example, the artificial intelligence 130 may be used to compare an image 120 to previous images from the historical data 112 to generate an output 140 of a change map. A change map shows the differences between previous images from the historical data 112 and the image 120 that is in the process of being acquired by the imaging device 110. In some examples, if the change map shows no evidence of changes between the previous images and image 120 that is in the process of being acquired by the imaging device 110, then the acquisition of image 120 that is in the process of being acquired by the imaging device 110 may be stopped in a short time. In some examples, if the change map shows evidence of differences between the previous images and the image 120 that is in the process of being acquired by the imaging device 110, then the imaging device 110 may continue with the acquisition of the image 120. In some examples, as a patient is imaged multiple times by an imaging system 100, multiple images are stored in the historical data 112, and the imaging system 100 may, for the creation of the output 140 of the change map, use any of the previous images from the historical data 112 to compare to the image 120 that is in the process of being acquired by the imaging device 110. Therefore, the artificial intelligence 130 allows for the imaging device 110 to gather only what is needed, reducing scanning time and computing resources. Change maps may also be used to assess the evolution of disease or response to therapy.

In another example, the artificial intelligence 130 may be used to generate an image with improved image quality (e.g., one or more of: increased spatial resolution, increased signal-to-noise ratio, increased contrast-to-noise ratio, image suitability in detection of a particular disease or process, etc.) as the output 140 To generate an improved image, the artificial intelligence 130 may use the historical data 112 and/or representation 114 to restore images that may be degraded, or of a lower initial quality, from less expensive and low-performing imaging systems to a higher quality expected from a more expensive, high-performance system. The artificial intelligence 130 may incorporate representations to fill in missing information, providing an improved image.

In a specific disease example of how an imaging system may be used, the imaging system 100 is tailored for an output 140 of an early detection of pancreatic cancer. Pancreatic cancer is an illness that is often detected too late in the progression of the illness for therapy to be effective. In the imaging system 100, the previous images from historical data 112 may be used to tailor acquisition by the imaging device 110 of new images 120 and sets of data 122 to hunt for subtle changes in pancreas features indicative of encroaching cancer. The artificial intelligence 130 may be configured to have been trained to identify changes in pancreas features between image 120 and a previous image from the historical data 112.

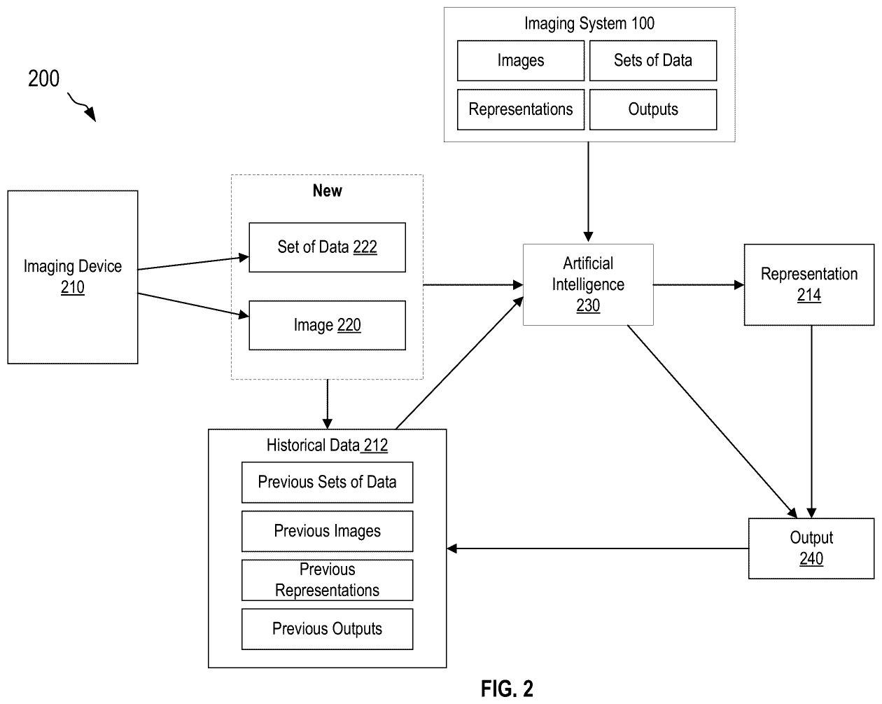

Referring to FIG. 2, an imaging system 200 is depicted according to an example embodiment. The imaging system 200 is a Type 2 imaging system that enables rapid screening of patients in non-specialty settings. Non-specialty settings may include nontraditional settings for advanced imaging, such as commercial settings like pharmacies or supermarkets, places of work, personal homes, or primary care physicians ‘offices. A Type 2 imaging system is trained on high-end images from a Type 1 imaging system and provides outputs 240 of targeted answers for non-experts. The imaging system 200 includes an imaging device 210, historical data 212, a representation 214, an image 220, a set of data 222, an artificial intelligence 230, data from an imaging system 100, and an output 240. The imaging system 200 is configured to use the artificial intelligence 230 to provide an image-informed early warning of changes in the health of a patient. The imaging system 200 may be used repeatedly over time for an individual patient.

Type 2 imaging systems (e.g., accessible scanners) have been developed in MRI, CT, ultrasound, and other imaging modalities. These systems are often aimed at regions and communities long underserved by imaging, and are also targeted to new point-of-care environments, like intensive care units and ambulances.

The imaging system 200 includes an imaging device 210 that is a stripped-down lower-performance scanner, such as a point-of-care scanner and/or a cheaper, more accessible medical imaging device. In some embodiments, the imaging device 210 is a low-field MRI machine, portable CT machine, and/or handheld ultrasound device. The imaging device 210 is a device that is configured to acquire images of a patient to be used in the imaging system 200. The image 220 is an image of a patient acquired via the imaging device 210.

The image 220, the set of data 222, the historical data 212, the artificial intelligence 230, the representation 214, and the output 240 of imaging system 200 are functionally and/or structurally similar to their respective components of imaging system 100 of FIG. 1.

In some examples, the image 220 may be an image acquired via MRI, CT, PET, Nuclear Imaging, ultrasound, and/or X-ray imaging, among other imaging modalities. A set of data 222 may be information related to an image 220 or may be another, separate image that shares the properties of an image 220. The set of data 222 may be acquired by a device that is separate from the imaging device 210 or the device may be the scanner 210 itself.

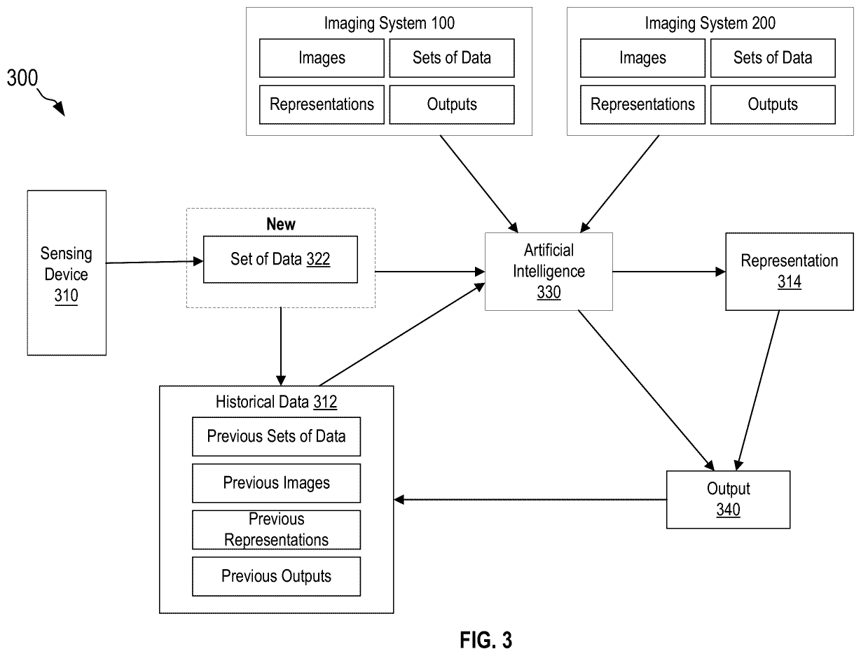

The imaging system 200 includes historical data 212. The historical data 212 includes previous information acquired by and/or created by the imaging system 200, such as by another Type 2 imaging system, and/or by a Type 1 imaging system 100, or Type 3 sensing system 300 (discussed further in reference in FIG. 3). For example, the historical data 212 may include previous images, sets of data, representations, and/or outputs. The artificial intelligence 230 is configured to use the historical data 212 to generate the representation 214, inform the acquisition of new images and/or set of data, and to allow for the detection of changes between images, sets of data, representations, and/or sub-combinations thereof over time.

The representation 214 is a model of an individual patient's baseline state of health. The representation 214 is a feature vector derived using the artificial intelligence 230, and generated from the image 220, and/or the set of data 222. The artificial intelligence 230 distills the image 220 and/or the set of data 222 into the representation 214. The representation 214 may be updated by the imaging system 200 when the patient undergoes a new imaging exam and a new image 220 and/or set of data 222 is acquired. An individual representation, or a cumulative set of representations generated over time may be used to assess risk, characterize change, improve image quality, and/or establish trajectories of health for a patient over time.

Multiple representations for an individual subject that vary over time are included in the historical data 212. As the historical data 212 allows for the usage of and comparisons to previous images, sets of data, and/or representations, updating a representation 214 with information from a new image 220 allows for the imaging system 200 to acquire a smaller quantity of time-consuming imaging data that makes up an image 220 than would otherwise be needed to generate a representation 214 in the absence of previous images from the historical data 212. The representations of the patient in the imaging system 200 may inform how the imaging system 200 is configured to acquire new images 220 and/or sets of data 222, and/or how the imaging system 200 is configured to generate new outputs 240.

The artificial intelligence 230 is configured to receive the image 220 or the set of data 222 to generate the representation 214 and output 240. The artificial intelligence is further configured to be trained on historical data 212, including images, sets of data, representations, and/or outputs from imaging system 200, as well as historical data 112 from imaging system 100. The artificial intelligence 230 may be stored as part of the imaging device 210 or on a separate computing device.

The artificial intelligence 230 may use various machine learning models to generate the representation 214 and output 240. In some embodiments, the machine learning model includes representation learning to reduce high-dimensional data to low-dimensional data, making it easier to discover patterns and anomalies. In some embodiments, representation learning is supervised, such as in a convolutional neural network. In some embodiments, representation learning is unsupervised, such as in an autoencoder network. In some embodiments, the artificial intelligence 230 may use a Siamese neural network structure. The artificial intelligence 230 provides the output 240. The output may include automated image interpretation, increased imaging speed, and/or improved imaging quality, among other outputs 240. The output 240 may then be provided back to the historical data 212 of the imaging device 210.

The output 240 may be change detection, in which the artificial intelligence 230 assesses the new image 220, the set of data 222, and/or the representation 214 in comparison to previous images, sets of data, and representations from the historical data 212 to flag changes in individual subjects and/or to identify early trajectories toward known diseases or development of risk indicators. In some examples, known diseases may include cancer or a neurodegenerative disease. This allows for automatic assessment to detect of change over time, and detection of subtle shifts that may not be discernible by a radiologist.

In another example, the artificial intelligence 130 may be used to compare an image 220 to previous images from the historical data 212 to generate an output 240 of a change map. A change map shows the differences between previous images from the historical data 212 and the image 220 that is in the process of being acquired by the imaging device 210. In some examples, if the change map shows no evidence of changes between the previous images and image 220 that is in the process of being acquired by the imaging device 210, then the acquisition of image 220 that is in the process of being acquired by the imaging device 210 may be stopped in a short time. In some examples, if the change map shows evidence of differences between the previous images and the image 220 that is in the process of being acquired by the imaging device 210, then the imaging device 210 may continue with the acquisition of the image 220. In some examples, as a patient is imaged multiple times by an imaging system 200, multiple images are stored in the historical data 212, and the imaging system 200 may, for the creation of the output 240 of the change map, use any of the previous images from the historical data 212 to compare to the image 220 that is in the process of being acquired by the imaging device 210. Therefore, the artificial intelligence 230 allows for the imaging device 210 to gather only what is needed, reducing scanning time and computing resources. Change maps may also be used to assess the evolution of disease or response to therapy.

In another example, the artificial intelligence 230 may generate an output 240 of an image with improved image quality. To generate an improved image, the artificial intelligence 230 may use the historical data 212 and/or representations 214 to restore images 220 that may be degraded to a higher quality more familiar from expensive, high performance machines as used in Type 1 imaging systems.

In another example, the artificial intelligence 230 may generate an output 240 of a clinical answer. The artificial intelligence 230 may be configured to have had end-to-end training of a single neural network to allow the imaging system 200 to go directly from raw data of an image 220 and/or a set of data 222 to an output 240 that is a clinical answer rather than an image. The clinical answer is an indicator of a concerning change and/or an index of suspicion. For example, a clinical answer may be a yes-or-no answer to indicate the presence or absence of prostate cancer when using the imaging device 210 for prostate imaging. In another example, a clinical answer may be a yes-or-no answer to indicate the presence or absence of cerebral bleeds when using the imaging device 210 for cerebral imaging. In some examples, in the event of a positive (e.g., “yes”) clinical answer, a healthcare provider may then direct the patient toward a specialist healthcare provider for Type 1 imaging from an imaging system 100. Therefore, non-imaging experts can provide effective, and more routine screening for diseases. The use of Type 2 imaging to apply artificial intelligence 230 to provide outputs 240 of clinical answers in place of images may facilitate the population-level screening of patients for the risk of known diseases such as prostate cancer or breast cancer, among others, with inexpensive imaging devices.

Referring to FIG. 3, a sensing system 300 is depicted according to an example embodiment. A sensing system 300 is a Type 3 imaging system that involves continuous health monitoring at the point of care, at work, and/or at home. A Type 3 sensing system 300 may be a stand-alone wearable and/or environmental sensor array and may be trained on information from Type 1 and/or Type 2 imaging systems and may then provide an early warning of changes. A Type 3 imaging system uses sensor data that is trained in the imaging settings of Type 1 and/or Type 2 imaging systems and is correlated with the imaging results of Type 1 and/or Type 2 imaging systems, with the Type 3 imaging system standing in place of advanced imaging for the purpose of early warning of disease. The sensing system 300 includes a sensing device 310, a set of data 322, an artificial intelligence 330, data from an imaging system 100 and/or an imaging system 200, and an output 340. The sensing system 300 may further include historical data 312 and/or a representation 314. The sensing system 300 is configured to use the artificial intelligence 330 to provide an image-informed early warning of changes in the health of a patient. The sensing system 300 may be used repeatedly over time for an individual patient.

The sensing system 300 includes a sensing device 310 that is a sensor and/or an array of sensors that is configured to acquire a set of data 322 of a patient. In some examples, the sensing device 310 may be a sensor for ultrasound, bioimpedance, electrocardiogram (EKG), electromyography (EMG), electroencephalography (EEG), radiofrequency (RF) pilot tone, ultra-wide band (UWB) radar, or near infrared (NIR), among other penetrating sensor modalities. In some examples, the sensing device 310 may be an accelerometer, optical camera, three-dimensional/time-of-flight (3D/TOF) camera, or skin conductance sensor, among other sensor modalities.

The sensors may be wearable, and monitor health continuously or at regular intervals. In some embodiments, the sensors may be incorporated into clothing or attached to a body. For example, a sensing system 300 may have a sensing device 310 that is a smart underwear sensor for an output 340 of a detection of prostate cancer, a smart bra sensor for an output 340 of a detection of a breast cancer, or another wearable or environmental sensor to monitor health states as an output 340.

The set of data 322, historical data 312, the representation 314, the artificial intelligence 330, and the output 340 of sensing system 300 are functionally and/or structurally similar to their respective components of imaging system 100 of FIG. 1 and imaging system 200 of FIG. 2.

The sensing system 300 may include historical data 312. The historical data 312 includes previous information acquired by and/or created by the sensing system 300, and/or by a Type 1 imaging system 100 and/or by a Type 2 imaging system 200. For example, the historical data 312 may include prior sensing device 310 generated sets of data, representations, and outputs, or prior sets of data, representations, or outputs from other sensing or imaging systems. The artificial intelligence 330 is configured to use the historical data 312 to generate the representation 314, inform the acquisition of new sets of data, and to allow for the detection of changes between images, sets of data, representations, and/or sub-combinations thereof over time.

The representation 314 is a model of an individual patient's baseline state of health. The representation 314 is a feature vector derived using the artificial intelligence 330, and generated from the set of data 322. The artificial intelligence 330 distills set of data 322 into the representation 314. The representation 314 may be updated by the sensing system 300 when a patient's sensing device acquires a new set of data 322. An individual representation, or a cumulative set of representations generated over time may be used to assess risk, characterize change, improve image quality, and/or establish trajectories of health for a patient over time.

The artificial intelligence 330 is configured to receive the set of data 322 to generate the representation 314 and output 340. The artificial intelligence may further configured to be trained on historical data 312, including images, sets of data, representations, and/or outputs from sensing system 300, as well as historical data 112 from imaging system 100 and historical data 212 from imaging system 200. The artificial intelligence 330 may be stored as part of the sensing device 310 or on a separate computing device.

The artificial intelligence 330 may use various machine learning models to generate the representation 314 and output 340. In some embodiments, the machine learning model includes representation learning to reduce high-dimensional data to low-dimensional data, making it easier to discover patterns and anomalies. In some embodiments, representation learning is supervised, such as in a convolutional neural network. In some embodiments, representation learning is unsupervised, such as in an autoencoder network. In some embodiments, the artificial intelligence 330 may use a Siamese neural network structure. The artificial intelligence 330 provides the output 340, and the artificial intelligence 330 may then provide the output 340 back to the historical data 312 of the sensing system 300 such that the sensing system 300 may use the historical data 312 to inform the acquisition of future sets of data 322 and/or to inform the future operation of an artificial intelligence 330.

The sensing system 300 provides indirect tomography through sensing device 310, trained through artificial intelligence 330 with high-end and/or low-end imaging machines from imaging systems 100 and/or imaging systems 200, to provide outputs 340 of spatial-resolved information about changes in tissue anatomy or function and/or early warnings of concerning changes in representations of patients’ states of health.

For example, the artificial intelligence 330 may be used for an output 340 of change detection, in which representations 314 are used to flag changes in individual subjects and/or to identify early trajectories toward known diseases or development of risk indicators. In change detection, the artificial intelligence 330 may compare a new set of data 322 to previous sets of data from the historical data 312 and report an output 340 of whether any differences have been determined between the new set of data 322 and the previous sets of data from the historical data 312. Similarly, the artificial intelligence 330 may compare a new representation 314 to a previous representation from the historical data 312 and report an output 340 of whether any differences have been determined between the new representation 314 and the previous representation from the historical data 312.

In another example, the artificial intelligence 330 may be used to compare the set of data 322 to previous sets of data from historical data 312 to generate an output 340 of a change map. A change map shows the differences between the set of data 322 and previous sets of data. In some examples, as the sensing device 310 acquires multiple sets of data for a patient in a sensing system 300, multiple sets of data are stored in the historical data 312 and the sensing system 300 may, for the creation of the output 340 of the change map, use any of the previously acquired sets of data from the historical data 312 to compare to the set of data 322 that is in the process of being acquired by the sensing device 310. Change maps may also be used to assess the evolution of disease or response to therapy.

The artificial intelligence 330 may be configured to have had end-to-end training of a single neural network to allow the sensing system 300 to go directly from raw data of a set of data 322 to an output 340 that is a clinical answer rather than a set of data. The clinical answer is an indicator of a concerning change and/or an index of suspicion. In some examples, in the event of a positive (e.g., “yes”) clinical answer, the sensing system 300 could then direct the patient toward a specialist healthcare provider for Type 1 imaging from an imaging system 100 and/or toward a non-specialist healthcare provider for Type 2 imaging from an imaging system 200. The use of Type 3 imaging to apply artificial intelligence 330 to provide outputs 340 of clinical answers facilitates the continuous health monitoring of patients at the point of care, at work, and/or at home, with inexpensive sensors of sensing device 310.

In another example, the sensing system 300 includes a sensing device 310 that is an article of smart clothing with wearable sensors that are configured to detect changes from a patient's baseline health or to provide a warning sign of a disease. For example, the sensing device 310 may be smart underwear, which is underwear that includes sensors configured to generate sets of data 322 continuously. The smart underwear may use the historical data 312 and/or representations that are derived from Type 1 imaging systems 100 and/or Type 2 imaging systems 200 to provide an output 340 that is a clinical answer on the presence or absence of an early stage of prostate cancer in the patient. In another example, the sensing device 310 may be a smart bra, which is a bra that includes sensors configured to generate sets of data 322 continuously. The smart bra may use the historical data 312 and/or representations that are derived from Type 1 imaging systems 100 and/or Type 2 imaging systems 200 to provide an output 340 that is a clinical answer on the presence or absence of an early stage of breast cancer in the patient. In another example, the sensing device 310 may be a smart hat, which is a hat that includes sensors configured to generate sets of data 322 continuously. The smart hat may use historical data 312 and/or representations that are derived from Type 1 imaging systems 100 and/or Type 2 imaging systems 200 to provide an output 340 that is a clinical answer of an assessment of the brain health of the patient.

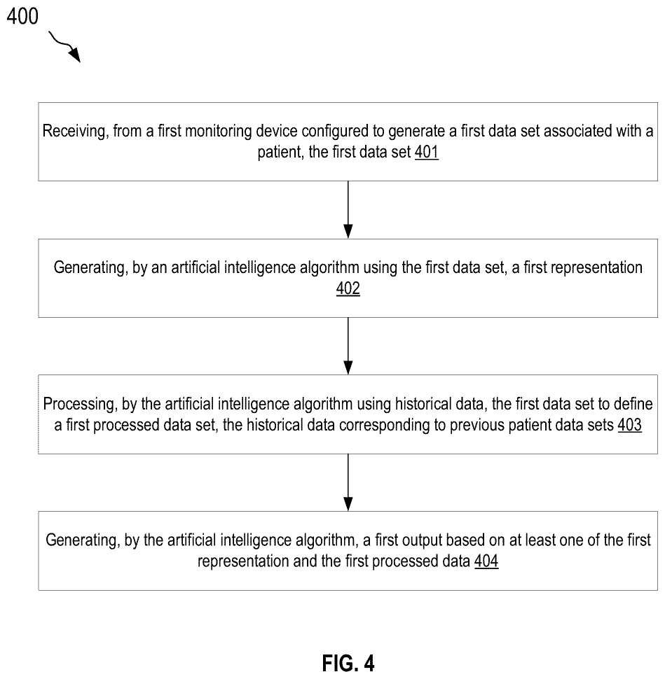

FIG. 4 is a method 400 for processing monitoring device data, according to an embodiment. The method 400 can be executed by at least one of the imaging system 100 of FIG. 1, the imaging system 200 of FIG. 2, and/or the sensing system 300 of FIG. 3. The method 400 includes receiving, from a first monitoring device configured to generate the first data set associated with a patient, the first data set at 401, generating, by an artificial intelligence algorithm using the first data set, a first representation at 402, processing, by the artificial intelligence algorithm using historical data, the first data set to define a first processed data set at 403, the historical data corresponding to pervious patient data sets, and generating, by the artificial intelligence algorithm, a first output based on at least one of the first representation and the first processed data at 404.

At 401, the method 400 includes receiving, from a first monitoring device configured to generate a first data set associated with a patient, the first data set. In some embodiments, the first monitoring device is an imaging device, such as the imaging device 110 of FIG. 1. In some embodiments, the first data set includes a set of data and an image.

At 402, the method 400 includes generating, by an artificial intelligence algorithm using the first data set, a first representation. In some embodiments, the artificial intelligence algorithm is functionally and/or structurally similar to the artificial intelligence 130 of FIG. 1. In some embodiments, the artificial intelligence algorithm includes representation learning. In some embodiments, at least one of a convolution neural network, a Siamese neural network, or an autoencoder network.

At 403, the method 400 includes processing, by the artificial intelligence algorithm using the first historical data, the first data set to define a first processed data set. The historical data corresponds to previous patient data sets, which may include images, sets of data, representations, and/or outputs. At 404, the method 400 includes generating a first output based on at least one of the first representation and the first processed data. In some embodiments, the output can include at least one of a clinical answer or a change map.

In some embodiments, the method 400 includes repeating 401, 402, and 403, with a second monitoring device (e.g., the imaging device 210), as described in reference to FIG. 2. The resulting second data set, second historical data, and second output being utilized by the artificial intelligence. In some embodiments, the method 400 includes additionally repeating 401, 402, and 403 with a third monitoring device (e.g., the sensing device 310), as described in reference to FIG. 3. The resulting third data set, third historical data, and third output being utilized by the artificial intelligence.

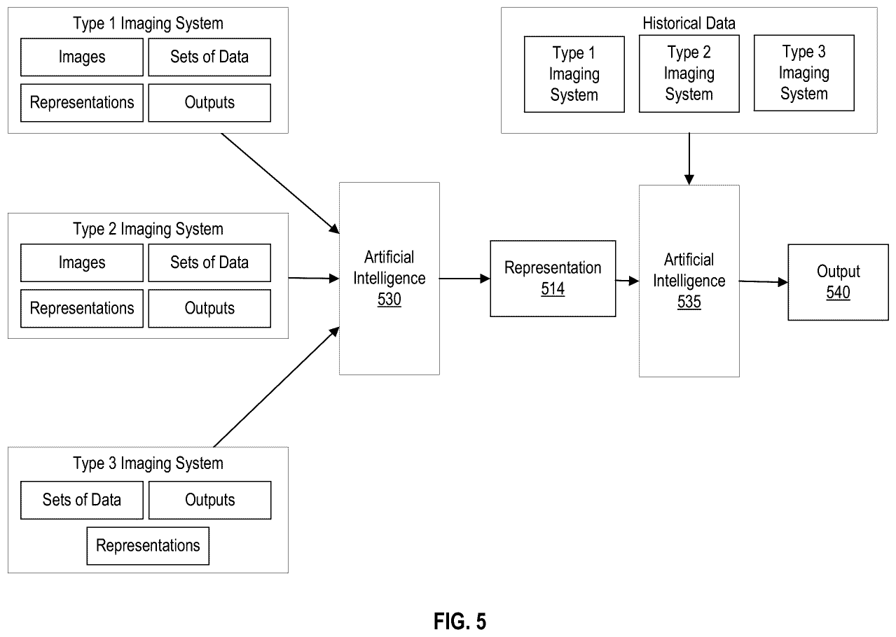

FIG. 5 illustrates an embodiment of a health diagnostic system wherein a Type 1, Type 2, and/or Type 3 system may provide a data set to a first artificial intelligence 530. The artificial intelligence 530 generates a representation of that data set corresponding to the one (or more) data sets from the respective imaging or sensing device at that respective time, such as a particular visit to a hospital. The representation 514 is analyzed by a second artificial intelligence 535, which may be different than the first artificial intelligence 530, such as a different algorithm or an entirely different neural network. The analysis of the representation 514 may utilize historical data from the Type 1, Type 2, and/or Type 3 systems to generate a new output 540. The output 540 may be an indication of a health diagnosis, such as but not limited to an indication of a risk of a condition and/or the change in the risk of a condition.

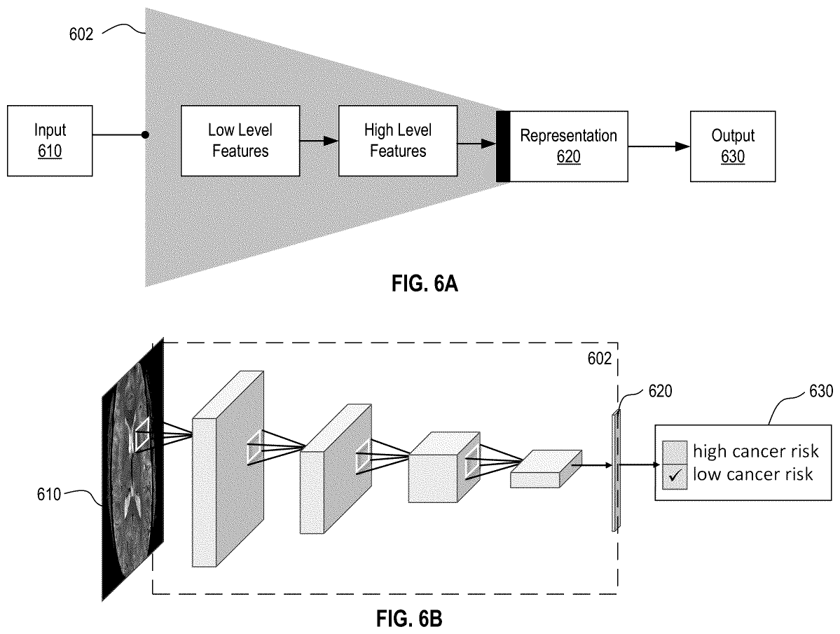

As discussed above with reference to FIGS. 1-5, artificial intelligence systems in the present invention may use neural networks. FIGS. 6A-6B illustrate the use of a convolutional neural network 602 used to generate an output 630 such as image classification, according to an embodiment. Referring to FIG. 6A, the convolutional neural network 602 distills input data 610, such as an image or signal, into progressively higher-level features, resulting in a lower-dimensional feature vector known as a representation 620. The representation 620 is used to generate an output 830. The input data 610 may include lower-level features such as edges and shading. The convolutional neural network 602 may include an encoder network that processes the input data 610 and compresses the input data 610 into the lower-dimensional representation 620 (e.g., feature vector). Referring to FIG. 6B, in some embodiments, the convolutional neural network 602 is used for classification, and the encoder network operates on the generated representation 620 to arrive at a discrete classification as an output 630. For example, in FIG. 6B, the output is an indication that the risk of cancer is low or high. The encoder network is trained using procedures such as backpropagation.

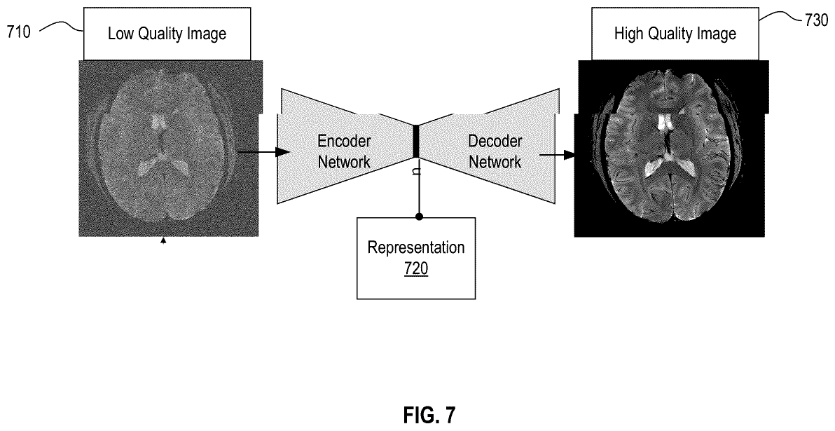

Referring to FIG. 7, a neural network architecture including an autoencoder network, commonly used for denoising, super-resolution, or other forms of image quality improvement, is shown. The autoencoder network includes an encoder network and a decoder network. The encoder network distills an input 710, such as low quality image, into a representation 720. Then, a decoder network converts the representation into a denoised/improved image. The output 730 is a high quality image. In some embodiments, the autoencoder network is trained with pairs of images, including low-quality images together with corresponding high-quality images. Autoencoders or other networks can be used to improve the quality of images obtained from low-performance devices, such as existing low-field MRI machines. In some embodiments, the autoencoder network may be used to improve image quality from user devices which that may be integrated into homes, places of work, retail/service businesses, or other public spaces (e.g., Type 2 imaging systems).

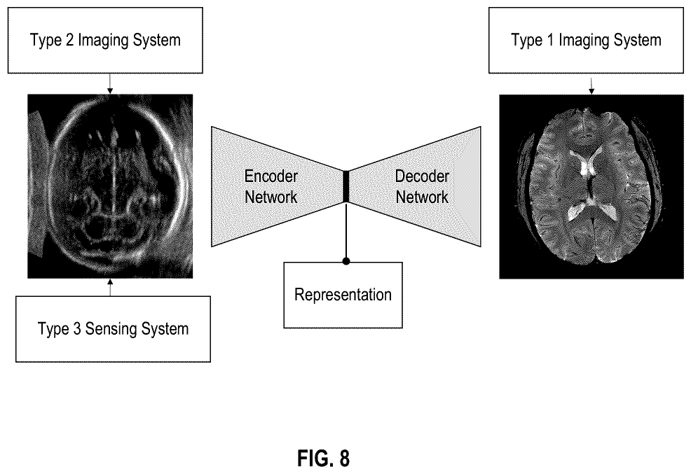

FIG. 8 illustrates an application of a neural network architecture including an autoencoder network for image quality transfer, in which one type of image is transformed to resemble the appearance of another type of image. An image quality transfer process makes a mapping between one characteristic image content or contrast to another (e.g., between an MR image and an image from a handheld ultrasound scanner, or a signal from a wearable or environmental sensor). The autoencoder network may be trained on paired images or signals (e.g., from the same person or object at the same/similar times). In some embodiments, the autoencoder network operates as a transformation network that can be used to convert imaging and/or sensing signals into a canonical form which may be used for comparison, classification, or other processing.

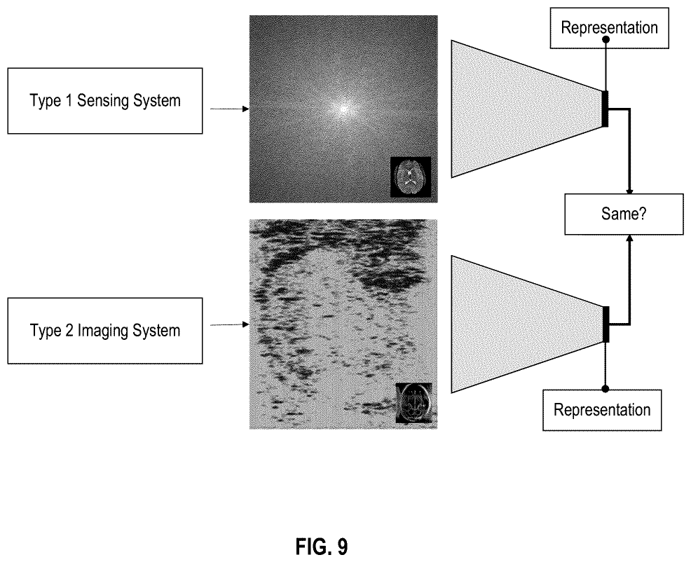

FIG. 9 illustrates an application of a neural network architecture including a Siamese network used for contrastive or self-supervised learning. A Siamese network comprises two component networks with shared weights, and can be trained to identify similarities between disparate inputs, such as images or signals. Siamese networks are able to extract features from each input and compare them to determine their similarity. An image or signal is fed into one branch of the Siamese network, and the other branch is fed with transformed versions of that image or signal. The resulting representations are compared.

This process is repeated for multiple images or signals and multiple transformations, and the Siamese network is trained to identify which transformed images or signals correspond to a given original image or signal, and which do not correspond. The Siamese network learns to generate representations which are similar for any transformed versions of the same image or signal, and different for transformed versions of different images or signals. The resulting trained Siamese network can recognize key similarities, ignoring irrelevant transformations such as rotations or translations (or whatever transformations are used during the training process).

In some embodiments, images or signals from different imaging or sensing modalities (e.g., MRI and ultrasound) obtained in the same person or object at similar times can be used as differently transformed versions of same thing, whereas images or signals in a different person or object, or a substantially changed person or object, can be used as examples of different things. A Siamese network trained with this input will establish a framework of shared representations connecting different imaging or sensing modalities. The Siamese network learns key similarities between high-quality imaging data, low-quality imaging data, and sensing data. In addition, the Siamese network learns to ignore irrelevant differences. When the weights are frozen after the training process, the common encoder component of the network can be used in isolation, and data entered into that encoder will be distilled into representations that capture key shared information among different data types.

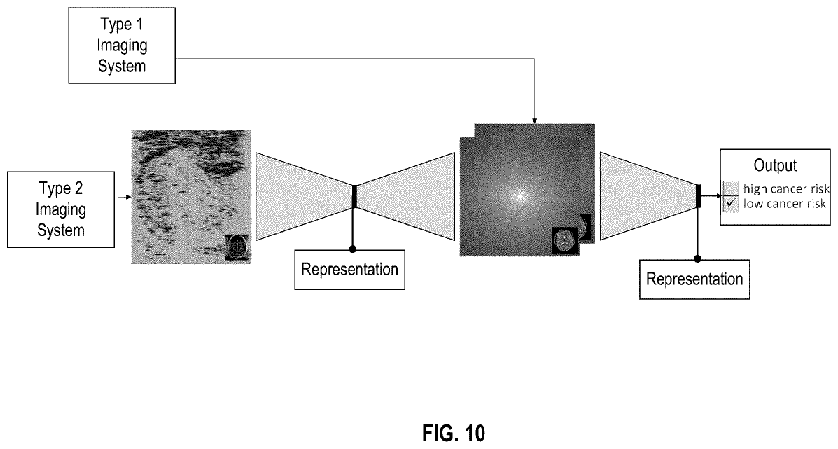

FIG. 10 illustrates an embodiment of the present disclosure in which data from one imaging or sensing modality is improved or transformed, using approaches based on shared representations (for example, shown in FIG. 9), such that the data resembles data from another imaging or sensing modality. In some embodiments, the resulting improved or transformed data is included as additional input into a classification network, resulting in improved classification as compared what may be obtained using a single imaging or sensing modality. As the additional input can take a consistent canonical form shared among imaging or sensing modalities, it can be incorporated conveniently.

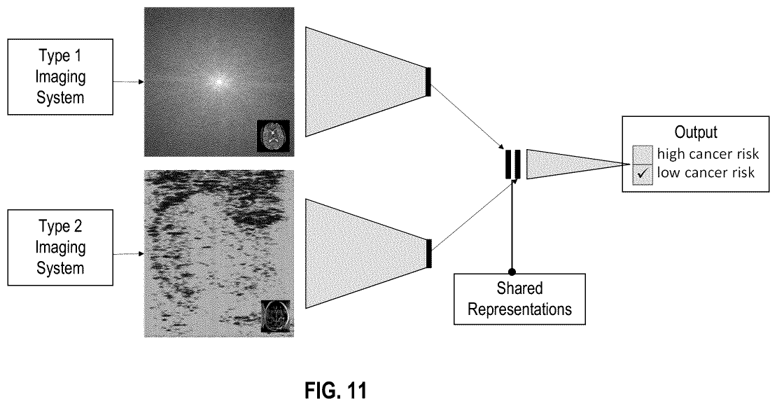

Shared representations may be used to refine risk assessment, optimize data acquisition, and generate improved images, combined images, and/or change maps. FIG. 11 illustrates an embodiment of the present disclosure in which a decoding step to create a canonical image or signal is eliminated. Data from different imaging or sensing modalities is distilled into shared representations using appropriately trained networks, and these representations are used directly as inputs into a classifier. In place of a classifier, other networks or algorithms can be used, with shared representations as inputs, to derive a wide range of parameters characterizing the structure or function of bodies, objects, or systems. This approach allows information from different imaging or sensing modalities to be combined into a common framework, yielding a combined and improved assessment of risk, or characterization of a state of health or functioning.

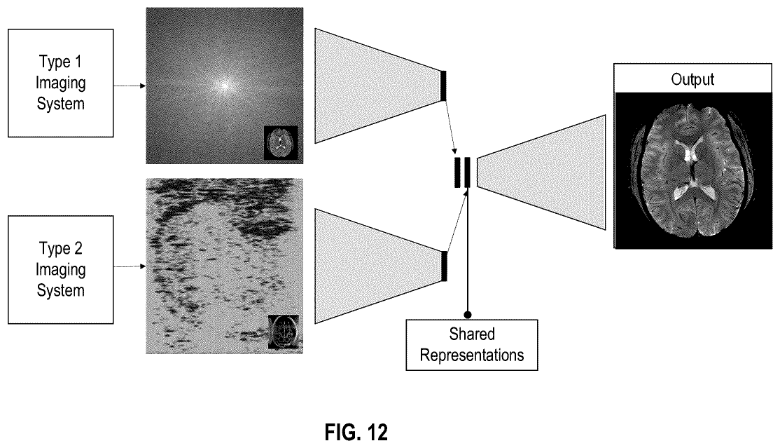

Referring to FIG. 12, shared representations may also be used to generate improved images, combined images, and/or difference maps. Incorporating representations from other imaging or sensing modalities can fill in information missing from a given modality, resulting in an improved image. Merging of shared representations can also be used as an alternative to traditional multimodality fusion methods generating visualizations of combined information. In some embodiments, decoder networks or other suitable networks can also be trained to highlight differences between information provided by different modalities, resulting in a detailed difference map. Although FIGS. 10-12 refer to combinations of pairs of signals or images, more than two sets of data from diverse imaging and sensing modalities may be combined in similar ways.

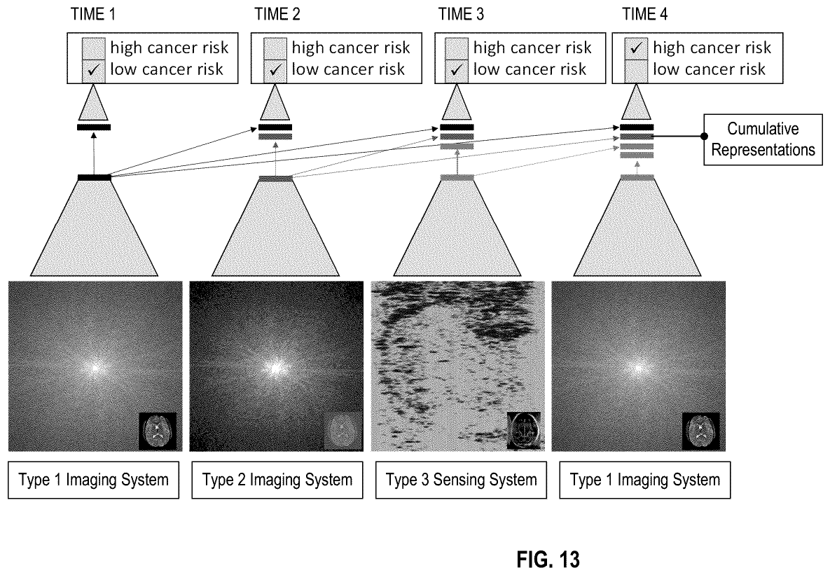

In addition to allowing combined use of information from multiple modalities, the use of shared representations further enables incorporation of information from distinct imaging or sensing sessions, regardless of the particular imaging or sensing modality. In some embodiments, representations generated from imaging or sensing data obtained at different times are combined, as illustrated in FIG. 13. As all the representations share a common framework, the representations can be used together to train classifiers for assessment of health risk, or other tasks. Each new representation can be added to a cumulative set of shared representations from previous imaging or sensing sessions, resulting in progressively improved assessments.

As data from multiple time points is present in such cumulative assessments, evaluations of a current state of health or functioning and changes from previous states are provided. The process shown in FIG. 13 reflects the goal of radiologists in looking at prior imaging examinations. For example, if a radiologist notes that a lesion or other abnormality is present in prior examinations, and that the appearance of such a lesion or abnormality is substantially unchanged from its appearance in previous examinations, the radiologist can generally rule the lesion out as an issue of immediate concern. In some embodiments, dynamic assessment of a current state can be performed automatically and quantitatively, using artificial intelligence.

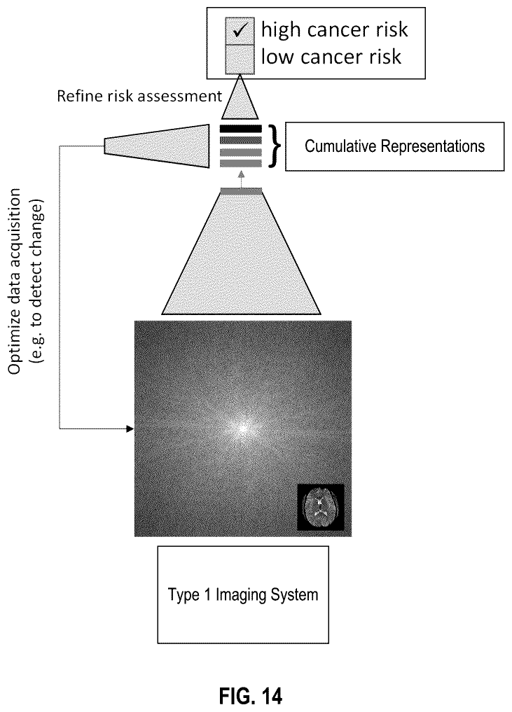

Shared representations can also be used beyond the applications of risk assessment or classification. Referring to FIG. 14, cumulative sets of representations can be used to modify data acquisition in a current imaging or sensing session. A neural network can be used to learn which datapoints are most important for detecting change, or for accomplishing other tasks of interest. For example, in MRI scans, shared representations can be used to choose which regions of data space, known as k-space, to acquire, which contrasts to probe, etc. in order to optimize the detection of change from previous imaging or sensing sessions.

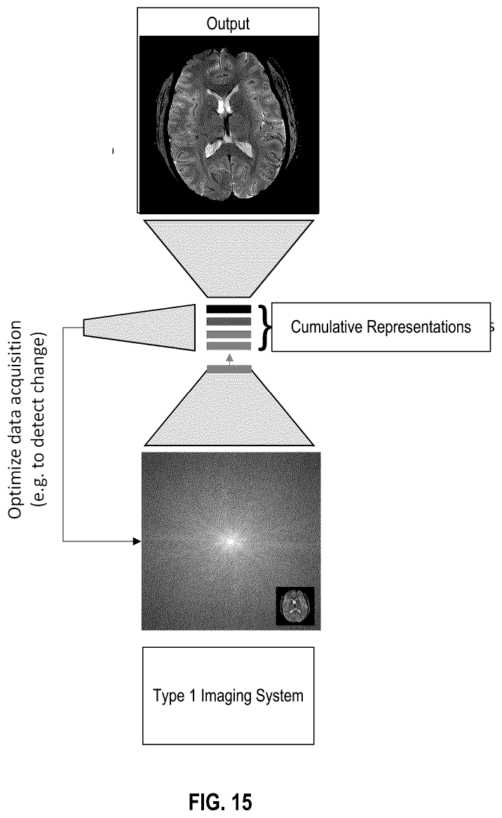

FIG. 15 illustrates an embodiment in which cumulative representations are used to generate improved images, combined images, and/or change maps. Incorporation of representations from other imaging or sensing sessions can be used to fill in information missing from a given session, resulting in improved image quality. Combining information from different imaging or sensing sessions provides an improved alternative to existing methods, such as simple juxtapositions or sequential displays on display media. In some embodiments, decoder networks or other suitable networks can also be trained to highlight differences between information gleaned during different imaging or sensing sessions, resulting in a detailed change map.

In medical applications, the generated change maps facilitate assessment of the evolution of disease or response to therapy. Radiologists, oncologists, neurologists and other physicians are often required to compare images obtained at different times, but typically do not have access to automated methods for identifying and depicting important changes. In some embodiments, the automated change maps generated from cumulative representations can be utilized beyond the healthcare arena, including in preventative maintenance, monitoring of satellite imagery, etc.

Referring to FIG. 11, multiple tiers or types (e.g., Type 1, Type 2, Type 3, etc.) of imaging or sensing technology can be linked together by AI-derived shared representations over time. As representations from different imaging or sensing sessions accumulate over time, information about health states is updated by available imaging or sensing modalities at each time point. Some of the imaging or sensing data may be derived from high-performance and limited-access machines, and other data may be derived from technologies which offer more limited performance or spatial resolution, but which afford broad, convenient and frequent access. In some embodiments, the resulting dynamic information regarding health states can be assembled into a health trajectory, which characterizes the baseline functioning of a body or other complex system, and which also flags noteworthy changes. In some embodiments, in addition to characterizing health trajectories in individuals, the present invention can be used to gather dynamic data from multiple individuals. This data may then be used to establish population norms. Population norms may be used to characterize the transition from health to disease (e.g., providing early signs of cancer, early indicators of the onset of metabolic disease, or subtle signs of encroaching dementia). Population norms established in this way can be used to identify concerning trends in individual health trajectories.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

The hardware and data processing components used to implement the various processes, operations, illustrative logics, logical blocks, modules and circuits described in connection with the embodiments disclosed herein may be implemented or performed with a general purpose single- or multi-chip processor, a graphical processing unit (GPU), a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates systems on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, erasable programmable read-only memory (EPROM), electronically erasable programmable read-only memory (EEPROM), or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

It is important to note that the construction and arrangement of the imaging systems as shown in the various exemplary embodiments is illustrative only. Additionally, any element disclosed in one embodiment may be incorporated or utilized with any other embodiment disclosed herein.

What is claimed is:

1. A method, comprising:
receiving input data, the input data comprising data from a plurality of modalities comprising imaging or sensing modalities;
processing the input data with a neural network trained to identify shared characteristics among the plurality of modalities, the neural network distilling the input data to generate a representation; and
processing the representation with the neural network to generate an output.

2. The method of claim 1, wherein the neural network includes an encoder network, the encoder network compressing the input data to generate the representation.

3. The method of claim 1, wherein the neural network includes an autoencoder network and the method further comprises training the autoencoder network using a set of image pairs, each image pair associated with a same subject and comprising a low-quality image and a corresponding high-quality image.

4. The method of claim 1, wherein the neural network includes a Siamese network.

5. The method of claim 1, wherein processing the input data comprises:
transforming data from a first modality using representations shared with a second modality; and
combining the transformed data from the first modality with data from the second modality to generate a common representation.

6. The method of claim 1, wherein:
the input data further comprises data acquired at different timepoints; and
processing the representation comprises processing representations generated from a prior timepoints a present timepoint.

7. The method of claim 6, further comprising forming a training set of cumulative representations by combining representations generated at different timepoints.

8. The method of claim 1, wherein the output is at least one of an image having improved image quality, a combined image, or a difference map.

9. The method of claim 1, wherein the output is a trajectory of function relative to a baseline.

10. The method of claim 9, further comprising transmitting a warning of undesired change in function.

11. A system, comprising:
a memory including instructions; and
at least one processor to execute the instructions to:
receive input data, the input data comprising at least one of:
data from multiple imaging modalities;
data from multiple sensing modalities; and
data from multiple different timepoints;
process the input data using a neural network to generate a representation; and
process the representation with the neural network to generate an output.

12. The system of claim 11, wherein the multiple imaging modalities comprise magnetic resonance imaging (MRI), computerized tomography (CT), positron emission tomography (PET), X-Ray, or ultrasound.

13. The system of claim 12, wherein the multiple imaging modalities comprise low-field MRI, portable CT, portable PET, or handheld ultrasound.

14. The system of claim 13, wherein the multiple imaging modalities comprise sensors.

15. The system of claim 11, wherein the output is image classification.

16. A non-transitory processor-readable medium storing code representing instructions to be executed by one or more processors, the instructions comprising code to cause the one or more processors to:
receive input data, the input data comprising at least one of:
data from multiple imaging modalities;
data from multiple sensing modalities; and
data from multiple different timepoints;
process the input data using a neural network to generate a representation; and
process the representation with the neural network to generate an output.

17. The non-transitory processor-readable medium of claim 16, wherein the neural network is an autoencoder network.

18. The non-transitory processor-readable medium of claim 16, wherein the output is image quality improvement.

19. The non-transitory processor-readable medium of claim 16, wherein the output is image quality transfer.

20. The non-transitory processor-readable medium of claim 16, wherein the instructions further cause the one or more processors to select or modify data acquisition using the neural network, wherein selecting or modifying data acquisition is based on improving change detection.
